(19) United States
(12) Patent Application Publication
(10) Pub. No.: US 2018/0044734 A1
(43) Pub. Date: Feb. 15, 2018

(54) COMPOSITION FOR DIAGNOSING SKIN DAMAGE CAUSED BY FINE DUST, AND COMPOSITION COMPRISING GALANGIN AS ACTIVE INGREDIENT

(71) Applicant: AMOREPACIFIC CORPORATION, Seoul (KR)

(72) Inventors: Hyoung-June KIM, Yongin-si, Gyeonggi-do (KR); Na Ri CHA, Yongin-si, Gyeonggi-do (KR); Ju Yearl PARK, Yongin-si, Gyeonggi-do (KR); Changjo JUNG, Yongin-si, Gyeonggi-do (KR); Lee-Kyoung KWON, Yongin-si, Gyeonggi-do (KR); Dong Wook SHIN, Yongin-si, Gyeonggi-do (KR); Tae Ryong LEE, Yongin-si, Gyeonggi-do (KR)

(21) Appl. No.: 15/554,886

(22) PCT Filed: Apr. 4, 2016
(86) PCT No.: PCT/KR2016/003464

§ 371 Date: Aug. 31, 2017

(30) Foreign Application Priority Data
Apr. 6, 2015 (KR) 10-2015-0048509
May 28, 2015 (KR) 10-2015-0074846
Apr. 1, 2016 (KR) 10-2016-0040355

(51) Int. Cl. C12Q 1/68; A61K 31/352; A23L 33/00; A61Q 19/00; A61Q 19/10; A23L 33/10; G01N 33/68; A61K 8/49

(52) U.S. Cl. CPC C12Q 1/6883; G01N 33/6893; A61K 31/352; A61K 8/498; A61Q 19/00; A61Q 19/10; A23L 33/10; A23L 33/30; C12Q 2600/136; C12Q 2600/158; G01N 2800/20; A23V 2002/00

(57) ABSTRACT

The present specification discloses a biomarker for diagnosing skin cell damage caused by fine dust, a kit using the same, and a novel use of a composition comprising galangin as an active ingredient. Specifically, it is possible to conveniently check skin damage caused by fine dust by measuring and comparing the expression amounts of the biomarker in normal cells and cells damaged by fine dust. In addition, a composition of the present invention comprising galangin, an isomer thereof, a pharmaceutically acceptable salt thereof, a prodrug thereof, a hydrate thereof or a solvate thereof as an active ingredient can normalize the gene expression of skin damaged by fine dust and promote differentiation of skin keratinocytes, thereby having a skin moisturizing or skin-barrier strengthening effect. Accordingly, since the composition can be used to prevent, treat, or alleviate skin diseases such as atopy, psoriasis, etc. the composition is useful.

### TECHNICAL FIELD

Disclosed in the present disclosure are a biomarker that can be used to diagnose skin damage by microdust, a composition comprising the same, a kit comprising the same and a novel use of a composition containing galangin as an effective ingredient.

### BACKGROUND ART

In skin, the epidermis plays an important role of preventing evaporation of water out of the human body. The epidermis is composed of the cornified layer, the granular layer, the spinous layer and the basal layer from outside. The cells of the cornified layer, or corneocytes, are embedded like bricks in a matrix of lipids which acts like mortar, thereby constituting the skin barrier (J. Invest. Dermatol. 80 (Suppl.), 44-49. 1983). In the corneocytes of a healthy person, natural moisturizing factors (NMFs) are present at high concentration, which help to moisturize skin. For example, water-soluble substances such as amino acids easily absorb water and prevent skin from drying (J. Invest. Dermatol. 54, 24-31, 1970).

However, for various reasons in terms of environments or life styles, including artificial temperature control through heating/cooling, skin stresses resulting from various social stress and environmental pollution, frequent face washing to remove makeup, chronological skin aging, etc., the skin surface becomes dry, rough, crumbly and lifeless due to decreased water content in the cornified layer. For this reason, the need of a skin moisturizer is increasing. Also, excessive physical and chemical stimulation from outside as well as UV, stress, malnutrition, etc. lead to decreased skin function and accelerate skin aging phenomena such as decreased elasticity, cornification, wrinkle formation, etc. In particular, the epidermis/dermis boundary is severely damaged by UV. Recently, increased pigmentation and nasolabial folds were observed in the skin of those who live in residential areas where yellow dust or fine dusts are severe.

### REFERENCES OF RELATED ART

### Non-Patent Documents

(Non-patent document 1) J. Invest. Dermatol. 80 (Suppl.), 44-49. 1983.

### DISCLOSURE

### Technical Problem

In an aspect, the present disclosure is directed to providing a method for diagnosing skin damage by microdust.

In another aspect, the present disclosure is directed to providing a biomarker that can be used to diagnose skin damage by microdust and a composition containing the same.

In another aspect, the present disclosure is directed to providing a composition which contains galangin as an effective ingredient.

In another aspect, the present disclosure is directed to providing a composition which is effective in moisturizing skin, enhancing skin barrier function or inducing differentiation of keratinocytes.

In another aspect, the present disclosure is directed to preventing or improving a skin disease associated with skin dryness or abnormality in skin barrier function.

In another aspect, the present disclosure is directed to providing a composition that can be used to improve skin damaged by microdust.

### Technical Solution

In an aspect, the present disclosure provides a composition for diagnosing damage of skin cells or skin barrier by microdust, which contains an agent for measuring the expression level of the mRNA of one or more gene selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27 (NM_006664) gene, IL8 (NM_000584) gene, PTGS2 (NM_000963) gene, NOX5 (NM_001184779) gene, XDH (NM_000379) gene, CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene and KRT13 (NM_002274) gene or protein thereof.

In another aspect, the present disclosure provides a kit for diagnosing damage of skin cells or skin barrier by microdust, which contains the composition.

In another aspect, the present disclosure provides a composition for moisturizing skin, which contains galangin, an isomer thereof, a pharmaceutically acceptable salt thereof, a prodrug thereof, a hydrate thereof or a solvate thereof as an effective ingredient.

In another aspect, the present disclosure provides a composition for enhancing skin barrier function, which contains galangin, an isomer thereof, a pharmaceutically acceptable salt thereof, a prodrug thereof, a hydrate thereof or a solvate thereof as an effective ingredient.

In another aspect, the present disclosure provides a composition for inducing differentiation of keratinocytes, which contains galangin, an isomer thereof, a pharmaceutically acceptable salt thereof, a prodrug thereof, a hydrate thereof or a solvate thereof as an effective ingredient.

In another aspect, the present disclosure provides a composition for improving skin damage by microdust, which contains galangin, an isomer thereof, a pharmaceutically acceptable salt thereof, a prodrug thereof, a hydrate thereof or a solvate thereof as an effective ingredient.

### Advantageous Effects

In an aspect, use of a biomarker for diagnosing skin cell damage by microdust and a composition containing the same enables convenient and quick diagnosis of skin cell damage by checking the expression level of genes whose expression is increased or decreased due to skin cell damage by microdust and allows for easy screening of an inhibitor of skin cell damage by microdust by investigating whether the activity of the proteins encoded by the genes is suppressed or increased.

In addition, a composition of the present disclosure which contains galangin, an isomer thereof, a pharmaceutically acceptable salt thereof, a prodrug thereof, a hydrate thereof or a solvate thereof as an effective ingredient can be used to prevent, treat or improve skin diseases such as atopy, psoriasis, etc. because it is effective in moisturizing skin or strengthening skin barrier by promoting the synthesis of filaggrin and keratin and promoting the differentiation of keratinocytes. Also, it may be used to improve skin damaged by microdust.

### BRIEF DESCRIPTION OF DRAWINGS

FIG. 1 shows cell viability after treatment with microdust extracts. ADSP denotes Asian dust storm particle, or yellow dust, PM10 (particulate matter 10) denotes microdust with a particle size of 10 μm and PM2.5 (particulate matter 2.5) denotes microdust with a particle size of 2.5 μm.

FIGS. 2a-2k show decreased expression of genes whose expression is increased in skin cells stimulated by microdust, after treatment with galangin.

FIGS. 3a-3k show increased expression of genes whose expression is decreased in skin cells stimulated by microdust, after treatment with galangin.

FIGS. 4a-4e show relative mRNA expression levels of filaggrin, keratin 10, keratin 1, keratin 13 and keratin 15 in normal human keratinocytes, depending on the concentration of galangin.

FIG. 5 shows the degree of differentiation of keratinocytes not treated with microdust, depending on the concentration of galangin.

FIG. 6 shows the degree of synthesis of filaggrin protein in normal human keratinocytes not treated with microdust, depending on the concentration of galangin.

### BEST MODE

Hereinafter, the present disclosure is described in detail.

The term “microdust” used in the present disclosure refers to particulate matter invisible to human eyes and floating or fluttering in the atmosphere for a long time. It may refer to particulate matter with a particle diameter of 10 μm or smaller. In particular, particulate matter with a particle diameter of 2.5 μm or smaller is called “ultrafine dust”. In the present disclosure, the term “microdust” is intended to include “ultrafine dust”.

The present disclosure relates to a biomarker for diagnosing skin cell damage or skin barrier damage by microdust, which contains one or more specific gene or a protein encoded by the gene.

The specific gene may be one or more gene selected from a group consisting of S100A7 (NM_002963), S100A8 (NM_002964), S100A9 (NM_002965), CYP1A1 (NM_000499), CYP1B1 (NM_000104), PI3 (NM_002638), IL36G (NM_019618), IL1B (NM_000576), CCL27 (NM_006664), IL8 (NM_000584), PTGS2 (NM_000963), NOXS (NM_001184779), XDH (NM_000379), CXCL14 (NM_004887), SOD3 (NM_003102), KRT1 (NM_006121), H19 (NR_002196), CASP14 (NM_012114), KRT10 (NM_000421), CASP8 (NM_001080125), KRT15 (NM_002275) and KRT13 (NM_002274).

One or more, specifically two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more or twenty two or more, of the genes or all of the genes may be used as a biomarker for diagnosing skin cell damage or skin barrier damage by microdust.

In another aspect, the present disclosure relates to a composition for diagnosing damage of skin cells or skin barrier by microdust, which contains an agent for measuring the expression level of the mRNA or protein of one or more gene selected from a group consisting of the above-described genes.

In another aspect, the present disclosure relates to a use of an agent for measuring the expression level of the mRNA of one or more gene selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27 (NM_006664) gene, IL8 (NM_000584) gene, PTGS2 (NM_000963) gene, NOX5 (NM_001184779) gene, XDH (NM_000379) gene, CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene and KRT13 (NM_002274) gene or protein encoded by the genes in preparation of a composition for diagnosing skin cell damage or skin barrier damage by microdust.

In another aspect, the present disclosure relates to a use of an agent for measuring the expression level of the mRNA of one or more gene selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27 (NM_006664) gene, IL8 (NM_000584) gene, PTGS2 (NM_000963) gene, NOX5 (NM_001184779) gene, XDH (NM_000379) gene, CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene and KRT13 (NM_002274) gene or protein encoded by the genes for diagnosis of skin cell damage or skin barrier damage by microdust.

In another aspect, the present disclosure relates to a method for diagnosing skin cell damage or skin barrier damage by microdust using an agent for measuring the expression level of the mRNA of one or more gene selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27

(NM_006664) gene, IL8 (NM_000584) gene, PTGS2 (NM_000963) gene, NOX5 (NM_001184779) gene, XDH (NM_000379) gene, CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene and KRT13 (NM_002274) gene or protein encoded by the genes.

The agent may be a polynucleotide complementary to the mRNA of the gene or a fragment thereof, a probe or a primer capable of amplifying the gene, or an antibody, e.g., a monoclonal antibody or a polyclonal antibody, specifically recognizing the protein.

In another aspect, the present disclosure relates to a kit which contains the composition for diagnosing damage of skin cells or skin barrier by microdust. By using the kit according to the present disclosure, skin cell damage or skin barrier damage by microdust can be diagnosed quickly and conveniently.

In an exemplary embodiment, the kit may be used to determine that skin is damaged by microdust 1) when the expression level of the mRNA of one or more gene selected from a group consisting of CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene, KRT13 (NM_002274) gene and filaggrin gene or protein encoded thereby measured from the skin cells of a subject is lower than that of a skin cell sample not damaged by microdust or 2) when the expression level of the mRNA of one or more gene selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27 (NM_006664) gene, IL8 (NM_000584) gene, PTGS2 (NM_000963) gene, NOX5 (NM_001184779) gene and XDH (NM_000379) gene or protein encoded thereby measured from the skin cells of a subject is higher than that of a skin cell sample not damaged by microdust.

In an exemplary embodiment, the kit may contain one or more antibody specifically recognizing the protein encoded by one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more or twenty two or more of the genes selected from a group consisting of S100A7, S100A8, S100A9, CYP1A1, CYP1B1, PI3, IL36G, IL1B, CCL27, IL8, PTGS2, NOX5, XDH, CXCL14, SOD3, KRT1, H19, CASP14, KRT10, CASP8, KRT15 and KRT13 or all of the genes, and skin damage by microdust may be determined by measuring the amount of antigens bound to the antibody in the skin cells of a subject.

In another aspect, the present disclosure relates to a method for diagnosing damage of skin cells or skin barrier by microdust. Specifically, the method may include: a) a step of measuring the expression level of the mRNA of one or more gene selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27 (NM_006664) gene, 1L8 (NM_000584) gene, PTGS2 (NM_000963) gene, NOX5 (NM_001184779) gene, XDH (NM_000379) gene, CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene and KRT13 (NM_002274) gene or protein encoded thereby from a skin cell sample of a subject; and b) a step of comparing the expression level with the expression level of the mRNA of the gene or protein encoded thereby in a skin cell sample not damaged by microdust.

In this aspect, the method may further include: a step of diagnosing that skin cells or skin barrier is damaged by microdust 1) when the expression level of the mRNA of one or more gene selected from a group consisting of CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene, KRT13 (NM_002274) gene and filaggrin gene or protein encoded thereby measured from the skin cells of a subject is lower than that of a skin cell sample not damaged by microdust or 2) when the expression level of the mRNA of one or more gene selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27 (NM_006664) gene, IL8 (NM_000584) gene, PTGS2 (NM_000963) gene, NOX5 (NM_001184779) gene and XDH (NM_000379) gene or protein encoded thereby measured from the skin cells of a subject is lower than that of a skin cell sample not damaged by microdust.

In this aspect, the expression level of the mRNA or protein of the gene may be measured by one or more method selected from a group consisting of microarray, PCR, NGS (nest-generation sequencing), western blot, northern blot, ELISA, radioimmunoassay, radioimmunodiffusion, histological immunostaining and immunoprecipitation assay.

As used in the present disclosure, the “normal level” of gene expression, etc. refers to the gene expression level in normal skin cells not stimulated by microdust. In the present disclosure, skin cell damage is diagnosed by measuring the amount of the mRNA of the gene or protein thereof in the skin cells of a subject and comparing it with the expression level of the mRNA of the gene or protein thereof in normal skin cells not stimulated by microdust.

As used in the present disclosure, the term “more” or “less” means that there is difference from the reference amount by 1.5 times or more or 2 times or more, specifically 2.2 times or more.

The genes used in the present disclosure whose expression is increased or decreased by microdust are described in Tables 1 and 2. Table 1 show the genes whose expression is increased by microdust and Table 2 show the genes whose expression is decreased by microdust. In the tables, name denotes the NCBI GenBank accession ID, gene symbol denotes the official symbol of the gene, and gene title denotes the name of the gene.

TABLE 1
Increased genes

| | Gene | |
|---|---|---|
| Name | symbol | Gene title |
| NM_002963 | S100A7 | S100 calcium binding protein A7 |
| NM_002964 | S100A8 | S100 calcium binding protein A8 |
| NM_002965 | S100A9 | S100 calcium binding protein A9 |
| NM_000499 | CYP1A1 | Cytochrome P450, family 1, subfamily A, |
| | | polypeptide 1 |
| NM_000104 | CYP1B1 | Cytochrome P450, family 1, subfamily B, |
| | | polypeptide 1 |
| NM_002638 | PI3 | Peptidase inhibitor 3, skin-derived |
| NM_019618 | IL36G | Interleukin 36, gamma |
| NM_000576 | IL1B | Interleukin 1, beta |
| NM_006664 | CCL27 | Chemokine (C-C motif) ligand 27 |
| NM_000584 | IL8 | Interleukin 8 |
| NM_000963 | PTGS2 | Cyclooxygenase-2 (COX-2) |
| NM_001184779 | NOX5 | NADPH oxidase, EF-hand calcium |
| | | binding domain 5 |
| NM_000379 | XDH | Xanthine dehydrogenase |

TABLE 2
Decreased genes

| | Gene | |
|---|---|---|
| Name | symbol | Gene title |
| NM_004887 | CXCL14 | Chemokine (C—X—C motif) ligand 14 |
| NM_003102 | SOD3 | Superoxide dismutase 3, extracellular |
| NM_006121 | KRT1 | Keratin 1 |
| NR_002196 | H19 | H19, imprinted maternally expressed |
| | | transcript |
| NM_012114 | CASP14 | Caspase 14, apoptosis-related |
| | | cysteine peptidase |
| NM_000421 | KRT10 | Keratin 10 |
| NM_001080125 | CASP8 | Caspase 8, apoptosis-related |
| | | cysteine peptidase |
| NM_002275 | KRT15 | Keratin 15 |
| NM_002274 | KRT13 | Keratin 13 |

The polynucleotide used as a probe in the kit of the present disclosure includes a full-length marker gene whose expression is increased or decreased by stimulation by microdust or a fragment thereof. Specifically, the fragment may be 10 nucleotides or longer. If the probe is 10 bps or shorter, it may bond nonspecifically.

The polynucleotide used as a primer in the kit of the present disclosure may be specifically 18-22 bps in length, although not being specially limited thereto.

The monoclonal antibody against the polynucleotide encoded by the marker gene contained in the kit of the present disclosure may be prepared by a general monoclonal antibody preparation method.

The present disclosure also relates to a composition which inhibits or improves skin cell damage by microdust by regulating the expression level of specific genes in skin cells damaged by microdust to a normal level.

In the present disclosure, the genes in skin cells whose expression is affected by microdust include S100A7, S100A8, S100A9, CYP1A1, CYP1B1, PI3, IL36G, IL1B, CCL27, IL8, PTGS2, NOX5, XDH, CXCL14, SOD3, KRT1, H19, CASP14, KRT10, CASP8, KRT15, KRT13, etc. Because S100A7, S100A8, S100A9, CYP1A1, CYP1B1, PI3, IL36G, IL1B, CCL27, IL8, PTGS2, NOX5 and XDH are the genes whose expression is increased by microdust, skin cell damage can be inhibited by decreasing the expression level of the genes to a normal level. And, because CXCL14, SOD3, KRT1, H19, CASP14, KRT10, CASP8, KRT15 and KRT13 are the genes whose expression is decreased by microdust, skin cell damage can be inhibited by increasing the expression level of the genes to a normal level.

In another aspect, the present disclosure relates to a method for screening a substance improving skin damage by microdust, which includes: a step of treating skin cells with microdust; a step of treating the microdust-treated skin cells with a test substance; and a step of checking the expression level of the mRNA of one or more gene selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27 (NM_006664) gene, IL8 (NM_000584) gene, PTGS2 (NM_000963) gene, NOX5 (NM_001184779) gene, XDH (NM_000379) gene, CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene and KRT13 (NM_002274) gene or protein encoded by the genes in the skin cells treated with the test substance, before and after the treatment with the test substance.

In this aspect, the method may further include a step of: determining the test substance as a substance improving skin damage by microdust 1) when the expression level of the mRNA of one or more gene selected from a group consisting of CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene, KRT13 (NM_002274) gene and filaggrin gene or protein encoded by the genes is increased or 2) when the expression level of the mRNA of one or more gene selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27 (NM_006664) gene, IL8 (NM_000584) gene, PTGS2 (NM_000963) gene, NOX5 (NM_001184779) gene and XDH (NM_000379) gene or protein encoded by the genes is decreased after the treatment with the test substance as compared to before the treatment with the test substance.

In an exemplary embodiment, the skin cell may be a keratinocyte.

The substance which inhibits or improves skin cell damage or skin barrier damage by microdust, which has been screened by the above-described method, includes galangin, although not being limited thereto.

In another aspect, the present disclosure relates to a composition for moisturizing skin comprising galangin, an isomer thereof, a pharmaceutically acceptable salt thereof, a prodrug thereof, a hydrate thereof or a solvate thereof as an effective ingredient.

In another aspect, the present disclosure relates to a method for moisturizing skin comprising a step of administering galangin, an isomer thereof, a pharmaceutically acceptable salt thereof, a prodrug thereof, a hydrate thereof or a solvate thereof to a subject in need thereof.

In another aspect, the present disclosure relates to a use of galangin, an isomer thereof, a pharmaceutically acceptable salt thereof, a prodrug thereof, a hydrate thereof or a solvate thereof for moisturizing skin.

In another aspect, the present disclosure relates to a use of galangin, an isomer thereof, a pharmaceutically acceptable salt thereof, a prodrug thereof, a hydrate thereof or a solvate thereof in preparing a composition for moisturizing skin.

In the present disclosure, “galangin” refers to a type of flavonoid which is a yellow crystal in needle shape. Its chemical formula is C₁₅H₁₀O₅, the molecular weight is 270 and the melting point is 214-215° C. It can be obtained from propolis, Helichrysum aureonitens, lesser galangal (Alpinia officinarum), galangal rhizome, etc. Galangin is known to have antibacterial and antiviral activity and to inhibit the growth of breast tumor cells. The structure of galangin is shown in Chemical Formula 1.

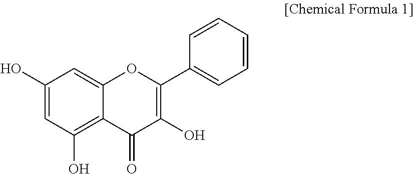 [SMILES: O=c1c(O)c(-c2ccccc2)oc2cc(O)cc(O)c12]

Galangin may have derivatives such as triacetylgalangin (C₁₅H₇O₂(OCOCH₃)₃) or trimethylgalangin (C₁₅H₇O₂(OCH₃)₃), although not being limited thereto.

As used in the present disclosure, an “isomer” includes not only optical isomers (e.g., essentially pure enantiomers, essentially pure diastereomers or mixtures thereof) but also conformation isomers (i.e., isomers which are different only in angles of one or more chemical bond), position isomers (especially, tautomers) or geometric isomers (e.g., cis-trans isomers).

In the present disclosure, “essentially pure” means, for example, when used in connection with enantiomers or diastereomers, that the specific compound as an example of the enantiomer or the diastereomer is present in an amount of about 90% (w/w) or more, specifically about 95% or more, more specifically about 97% or more or about 98% or more, further more specifically about 99% or more, even more specifically about 99.5% or more.

In the present disclosure, “pharmaceutically acceptable” means approved by a regulatory agency of the government or an international organization or listed in the Pharmacopoeia or other generally recognized pharmacopoeia for use in animals, more specifically in human, since significant toxic effect can be avoided when used with a common medicinal dosage.

In the present disclosure, a “pharmaceutically acceptable salt” refers to a salt according to an aspect of the present disclosure which is pharmaceutically acceptable and has a desired pharmacological activity of its parent compound. It includes a common salt formed from an inorganic acid, an organic acid, an inorganic base or an organic base and an acid addition salt of a quaternary ammonium ion. The salt may include: (1) an acid addition salt formed from an inorganic acid such as hydrochloric acid, hydrobromic acid, sulfuric acid, nitric acid, phosphoric acid, etc. or an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tert-butylacetic acid, lauryl sulfuric acid, gluconic acid, glutamic acid, hydroxynaphthoic acid, salicylic acid, stearic acid or muconic acid; or (2) a salt formed when an acidic proton present in the parent compound is substituted.

In the present disclosure a “prodrug” refers to a drug whose physical and chemical properties have been changed such that it does not exhibit physiological activity as it is but exerts medicinal effect after it is converted to the original drug through chemical or enzymatic action in vivo.

In the present disclosure, a “hydrate” refers to a compound to which water is bound. The term is used in a broad concept, including an inclusion compound which lacks chemical bonding between water and the compound.

In the present disclosure, a “solvate” refers to a higher-order compound formed between a solute molecule or ion and a solvent molecule or ion.

In another aspect, the present disclosure relates to a composition for enhancing skin barrier function comprising galangin, an isomer thereof, a pharmaceutically acceptable salt thereof, a prodrug thereof, a hydrate thereof or a solvate thereof as an effective ingredient.

In another aspect, the present disclosure relates to a method for enhancing skin barrier function comprising a step of administering galangin, an isomer thereof, a pharmaceutically acceptable salt thereof, a prodrug thereof, a hydrate thereof or a solvate thereof to a subject in need thereof.

In another aspect, the present disclosure relates to a use of galangin, an isomer thereof, a pharmaceutically acceptable salt thereof, a prodrug thereof, a hydrate thereof or a solvate thereof in enhancing skin barrier function.

In another aspect, the present disclosure relates to a use of galangin, an isomer thereof, a pharmaceutically acceptable salt thereof, a prodrug thereof, a hydrate thereof or a solvate thereof in preparing a composition for enhancing skin barrier function.

In another aspect, the present disclosure relates to a composition for inducing differentiation of keratinocytes comprising galangin, an isomer thereof, a pharmaceutically acceptable salt thereof, a prodrug thereof, a hydrate thereof or a solvate thereof as an effective ingredient.

In another aspect, the present disclosure relates to a method for inducing differentiation of keratinocytes comprising a step of administering galangin, an isomer thereof, a pharmaceutically acceptable salt thereof, a prodrug thereof, a hydrate thereof or a solvate thereof to a subject in need thereof.

In another aspect, the present disclosure relates to a use of galangin, an isomer thereof, a pharmaceutically acceptable salt thereof, a prodrug thereof, a hydrate thereof or a solvate thereof in inducing differentiation of keratinocytes.

In another aspect, the present disclosure relates to a use of galangin, an isomer thereof, a pharmaceutically acceptable salt thereof, a prodrug thereof, a hydrate thereof or a solvate thereof in preparing a composition for inducing differentiation of keratinocytes.

In another aspect, the present disclosure relates to a composition for improving skin damage by microdust comprising galangin, an isomer thereof, a pharmaceutically acceptable salt thereof, a prodrug thereof, a hydrate thereof or a solvate thereof as an effective ingredient.

In the present disclosure, the term skin damage is used in a broad concept, including the decline or weakening of skin function. For example, it may include the decline in skin barrier function, decline in skin-moisturizing ability, decline in skin elasticity, etc.

In another aspect, the present disclosure relates to a method for improving the conditions of skin damaged by microdust comprising a step of administering galangin, an isomer thereof, a pharmaceutically acceptable salt thereof, a prodrug thereof, a hydrate thereof or a solvate thereof to a subject in need thereof.

In another aspect, the present disclosure relates to a use of galangin, an isomer thereof, a pharmaceutically acceptable salt thereof, a prodrug thereof, a hydrate thereof or a solvate thereof in improving skin damage by microdust.

In another aspect, the present disclosure relates to a use of galangin, an isomer thereof, a pharmaceutically acceptable salt thereof, a prodrug thereof, a hydrate thereof or a solvate thereof in preparing a composition for improving skin damage by microdust.

The composition according to an aspect of the present disclosure may contain 0.000001-30 wt % of galangin, an isomer thereof, a pharmaceutically acceptable salt thereof, a prodrug thereof, a hydrate thereof or a solvate thereof based on the total weight of the composition. When the content is 0.000001-30 wt %, superior effect of moisturizing skin, enhancing skin barrier function, inducing differentiation of keratinocytes, etc. can be achieved.

Specifically, the content may be 0.0000001 wt % or more, 0.0000005 wt % or more, 0.0000007 wt % or more, 0.0000009 wt % or more, 0.000001 wt % or more, 0.000002 wt % or more, 0.000004 wt % or more, 0.000006 wt % or more, 0.000008 wt % or more, 0.00001 wt % or more, 0.00003 wt % or more, 0.00005 wt % or more, 0.00007 wt % or more, 0.00009 wt % or more, 0.0001 wt % or more, 0.0003 wt % or more, 0.0005 wt % or more, 0.0007 wt % or more, 0.0009 wt % or more, 0.001 wt % or more, 0.01 wt % or more, 0.1 wt % or more, 1 wt % or more, 3 wt % or more, 5 wt % or more, 7 wt % or more, 9 wt % or more, 10 wt % or more, 13 wt % or more, 15 wt % or more, 17 wt % or more, 19 wt % or more, 21 wt % or more, 23 wt % or more, 25 wt % or more, 27 wt % or more, 29 wt % or more, 30 wt % or more or 31 wt % or more and may be 32 wt % or less, 31 wt % or less, 30 wt % or less, 29 wt % or less, 28 wt % or less, 26 wt % or less, 24 wt % or less, 22 wt % or less, 20 wt % or less, 18 wt % or less, 16 wt % or less, 14 wt % or less, 12 wt % or less, 10 wt % or less, 9 wt % or less, 8 wt % or less, 6 wt % or less, 4 wt % or less, 2 wt % or less, 1 wt % or less, 0.1 wt % or less, 0.09 wt % or less, 0.04 wt % or less, 0.01 wt % or less, 0.006 wt % or less, 0.001 wt % or less, 0.0009 wt % or less, 0.0007 wt % or less, 0.00005 wt % or less, 0.00003 wt % or less, 0.00001 wt % or less, 0.000009 wt % or less, 0.000007 wt % or less, 0.000005 wt % or less, 0.000003 wt % or less, 0.000001 wt % or less, 0.0000009 wt % or less, 0.0000007 wt % or less, 0.0000005 wt % or less, 0.0000003 wt % or less, 0.0000002 wt % or less, 0.0000001 wt % or less or 0.00000009 wt % or less, although not being limited thereto.

In this aspect, the concentration of the galangin, the isomer thereof, the pharmaceutically acceptable salt thereof, the prodrug thereof, the hydrate thereof or the solvate thereof may be 0.1-5 μM based on the total volume of the composition.

Specifically, the concentration may be 0.1 μM or higher, 0.2 μM or higher, 0.3 μM or higher, 0.4 μM or higher, 0.45 μM or higher, 0.47 μM or higher, 0.49 μM or higher, 0.5 μM or higher, 0.51 μM or higher, 0.53 μM or higher, 0.55 μM or higher, 0.6 μM or higher, 0.7 μM or higher, 0.8 μM or higher, 0.9 μM or higher, 1.0 μM or higher, 1.1 μM or higher, 1.2 μM or higher, 1.3 μM or higher, 1.5 μM or higher, 1.7 μM or higher, 1.9 μM or higher, 2.0 μM or higher, 2.1 μM or higher, 2.3 μM or higher, 2.5 μM or higher, 2.7 μM or higher, 2.9 μM or higher, 3.0 μM or higher, 4.0 μM or higher, 4.5 μM or higher, 5.0 μM or higher or 5.1 μM or higher and may be 5.1 μM or lower, 4.6 μM or lower, 4.1 μM or lower, 3.6 μM or lower, 3.1 μM or lower, 2.6 μM or lower, 2.3 μM or lower, 2.2 μM or lower, 2.1 μM or lower, 2.0 μM or lower, 1.9 μM or lower, 1.8 μM or lower, 1.6 μM or lower, 1.4 μM or lower, 1.2 μM or lower, 1.1 μM or lower, 1.0 μM or lower, 0.9 μM or lower, 0.8 μM or lower, 0.6 μM or lower, 0.5 μM or lower, 0.4 μM or lower, 0.3 μM or lower or 0.2 μM or lower, although not being limited thereto. The effect of the composition may be better when the concentration is 0.2 μM or higher.

In this aspect, the composition may promote the expression of one or more selected from a group consisting of CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene, KRT13 (NM_002274) gene and filaggrin gene. Also, the composition may promote the synthesis of filaggrin protein or keratin protein.

And, the composition may decrease the expression of one or more selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27 (NM_006664) gene, IL8 (NM_000584) gene, PTGS2 (NM_000963) gene, NOXS (NM_001184779) gene and XDH (NM_000379) gene.

Accordingly, the composition according to an aspect of the present disclosure exhibits superior effect in preventing, improving or treating atopic dermatitis, psoriasis, xerotic dermatitis, etc.

In an aspect of the present disclosure, the composition may be a cosmetic composition, a pharmaceutical composition or a health functional food composition.

The cosmetic composition may be, for example, a cream, a lotion, etc. a cleanser, a facial cleanser, a soap, a cosmetic solution, etc.

A cosmetic product to which the galangin-containing composition of the present disclosure is added may be in the form of a solution, an emulsion, a viscous mixture, etc.

The cosmetic product of the present disclosure is not particularly limited in terms of formulation. For example, it may be formulated as an emulsion, a cream, a toilet water, an essence, a pack, a gel, a powder, a makeup base, a foundation, a lotion, an ointment, a patch, a cosmetic solution, a cleansing foam, a cleansing cream, a cleansing water, a body lotion, a body cream, a body oil, a body essence, a shampoo, a rinse, a body cleanser, a soap, a hair dye, a spray, etc.

Each formulation of the cosmetic composition may contain ingredients other than the galangin, which can be selected by those skilled in the art without difficulty depending on the particular formulation or purpose of use.

The formulation may contain a skin absorption-promoting material in order to increase the effect of moisturizing skin, enhancing skin barrier function and inducing differentiation of keratinocytes.

Also, the cosmetic formulation of the present disclosure may include one or more selected from a group consisting of a water-soluble vitamin, an oil-soluble vitamin, a polypeptide, a polysaccharide, a sphingolipid and a seaweed extract.

The cosmetic formulation of the present disclosure may contain, in addition to the essential ingredients, other ingredients commonly used in cosmetics.

Examples of the further added ingredients may include an oil, a fat, a humectant, an emollient, a surfactant, an organic or inorganic pigment, an organic powder, a UV absorbent, an antiseptic, a sterilizer, an antioxidant, a plant extract, a pH control agent, an alcohol, a colorant, a fragrance, a blood circulation stimulant, a cooling agent, an antiperspirant, purified water, etc.

However, the ingredients that can be added are not limited thereto. And, the amount of the further added ingredients may be determined within the range not negatively affecting the purpose and effect of the present disclosure.

The pharmaceutical composition comprising galangin of the present disclosure may further comprise a suitable carrier, excipient and diluent commonly used in preparing a pharmaceutical composition.

The pharmaceutical composition comprising galangin according to the present disclosure may be formulated into any pharmaceutically suitable formulation including an ointment, a gel, a cream, a patch, a spray, etc. according to common methods.

The administration dosage of the formulation may be 1.0-3.0 mL/day although it varies depending on the age, sex, body weight and symptoms of a subject and administration method. Specifically, the administration may be made 1-5 times a day for one month or longer.

The health food may refer to a food prepared using nutrients or functional ingredients that may lack in daily diets, which can maintain and improve health by maintaining the normal function of the human body or activating physiological functions, although not being limited thereto. The health food may be prepared and processed into a tablet, a capsule, a powder, a granule, a liquid, a pill, etc., although not being limited thereto, in accordance with related laws.

In an aspect of the present disclosure, a health drink composition may further contain, in addition to the above-described compound as the essential ingredient, other ingredients such as various flavors, natural carbohydrates, etc. commonly used in drinks without particular limitation. Examples of the natural carbohydrate include common sugars such as a monosaccharide, a polysaccharide, a cyclodextrin, etc. and sugar alcohols such as xylitol, sorbitol, erythritol, etc. In addition, a natural flavor (thaumatin or stevia extract (e.g., rebaudioside A, glycyrrhizin, etc.)) or a synthetic flavor (saccharin, aspartame, etc.) may be used as the flavor.

In general, the administration dosage of the effective ingredient contained in the health food composition may be about 0.0001-1000 mg/kg/day. More specifically, the administration dosage may be 0.02-6 mg/kg/day. The administration may be made once or several times a day.

Hereinafter, the present disclosure will be described in detail through examples. However, the following examples are for illustrative purposes only and it will be apparent to those of ordinary skill in the art that the scope of the present disclosure is not limited by the examples.

### EXAMPLE 1

### Collection and Extraction of Microdust

Microdust was collected using a low-volume air sampler (Sensidyne, Gillian, Low Volume Air Sampler, FL, USA). Sampling was conducted for about 24 hours while replacing a filter and a denuder of a filter pack around 10 a.m. on the day when sampling was made. Microdust was collected every day from Feb. 1, 2014 until Feb. 28, 2014 in a downwind area of Seoul, Korea (Yongin City, on the rooftop of a six-story building). Sampling time was recorded by checking the time while a vacuum pump was operated using a timer. Sampling rate, which was set to 16.7 L/min, was measured when the sampling was started and finished using a flow meter (Model 4143, TSI Inc.). A Teflon filter loaded into the filter pack was weighed before and after the sampling. Before weighing the Teflon filter, it was settled for 24 hours in a desiccator (Nikko, Japan) of 40% relative humidity. The weight was measured twice using an electronic balance (DVG215CD, Ohaus) to the five digits to the right of the decimal point and then averaged. Also, after the sampling, the filter was weighed twice after settlement in a desiccator for 24 hours. Mass concentration was calculated from the weight measured before the sampling. Microdust was extracted as follows. The Teflon filter was soaked in 1 mL of ethanol. After adding 14 mL of DW so that the water level reached the aerosol sampling surface of the filter and capping, extraction was conducted for 30 minutes by sonication. After completely removing water from the filter in a desiccator for 48 hours to minimize error, the weight of the filter was measured using a high-precision balance (Mettler Toledo Company) which can measure up to 0.1 mg.

### EXAMPLE 2

### Culturing of Normal Human Keratinocytes

Normal human keratinocytes (epidermal neonatal keratinocyte cells) purchased from Lonza, Inc. (Walkersville, Md., USA) were cultured in a CO₂ incubator under the condition of 37° C. and 5% CO₂. The cells were cultured according to the instructions of Lonza, Inc. KGMTM-2 Bullet Kit CC-3107(ingredients: BPE(bovine pituitary extract), human epidermal growth factor(hEGF), insulin, hydrocortisone, transferrin, epinephrine, and GA-1000(gentamycin sulfate+amphotericin-B)) which added KGM-2 Bullet kit CC-4152 to 500 mL of KBM-2 (KBMTM-2, CC-3103) medium, were used.

### EXAMPLE 3

### Treatment of Normal Human Keratinocytes with Microdust and Measurement of Cytotoxicity

In order to investigate the cytotoxicity of microdust, MTT assay was conducted using normal human keratinocytes according to the Mossman et al.'s method (J. Immunol. Methods, 65, 55-63, 1983).

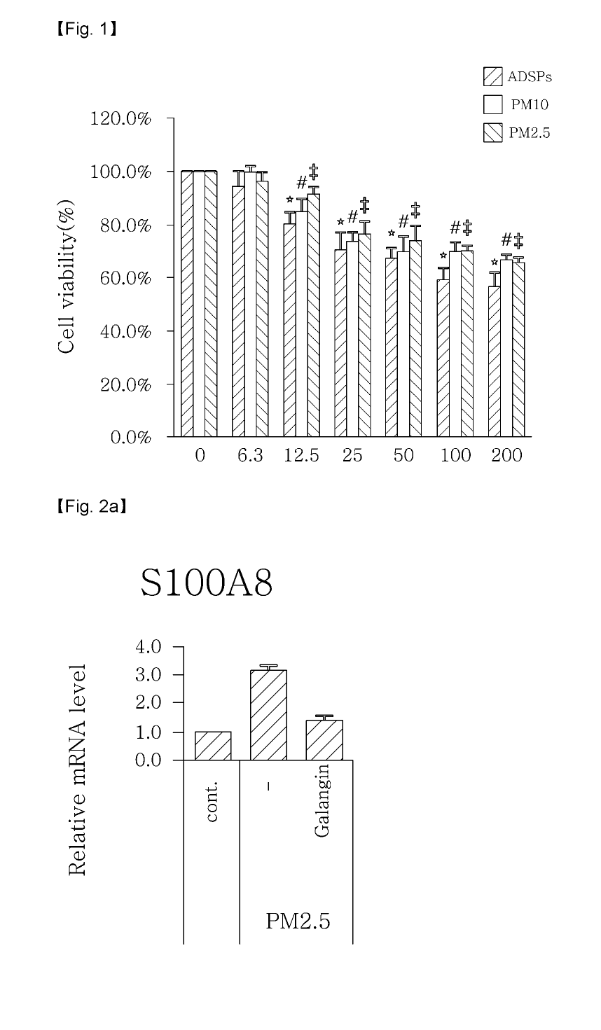

Specifically, a 24-well plate is used and microdust with a particle diameter of 10 μm and microdust with a particle diameter of 2.5 μm obtained in Example 1 was respectively dispersed in purified water. After treating 2.5×10⁵ normal human keratinocytes per cell, which were cultured under the condition of Example 2, with the prepared microdust dispersion, and culturing for 24 hours, the cells were further cultured at 37° C. for 3 hours after adding 5 mg/mL MTT (3-4,5-dimethylthiazol-2,5-diphenyltetrazolium bromide). Then, the medium was removed and the formed formazan crystal was dissolved in 500 μL of DMSO. The dissolved formazan crystal was transferred to a 96-well plate and OD value was determined by measuring absorbance at 540 nm. The result is shown in FIG. 1.

As seen from FIG. 1, for both the dispersions in which the microdust with a particle diameter of 10 μm and microdust with a particle diameter of 2.5 μm were dispersed (hereinafter, aqueous microdust extracts), the concentration at which cell survivability was 80% (10₂₀) was 12.5 μg/mL.

### EXAMPLE 4

### Analysis of Change in Genes in Cells by Microdust by Next-Generation Sequencing

For RNA-seq data processing and analysis, the general analysis method developed by Trapnell et al. (2012) was used. FastQC (http://www.bioinformatics.babraham.ac.uk/projects/fastqc/) was used for quality control of the RNA-seq data and FASTX (http://hannonlab.cshl.edu/fastx_toolkit/) was used to remove base and adaptor sequences of low accuracy. Then, alignment was performed using Tophat (Trapnell et al., 2009) and human genome (hg19) and the data quantity for each sample was confirmed using EVER-seq renamed as RSeQC (Wang et al., 2012). Also, the expression level of transcripts was quantified with Cufflinks and comparison was made between the samples treated with the two microdust dispersions and a normal sample (Trapnell et al., 2010). By applying a strict cutoff of FDR adjusted p-value<0.05 and ≧2.0 fold-change, the genes which showed significant change in expression upon treatment with the dispersion of microdust with a particle diameter of 2.5 μm and the dispersion of microdust with a particle diameter of 10 μm were determined. The result is shown in Tables 3 and 4.

TABLE 3
Increased genes

| Name | Gene symbol | Fold change |
|---|---|---|
| NM_002963 | S100A7 | 9.515833375 |
| NM_002964 | S100A8 | 3.766981583 |
| NM_002965 | S100A9 | 5.179254242 |
| NM_000499 | CYP1A1 | 48.06825714 |
| NM_000104 | CYP1B1 | 34.49696749 |
| NM_002638 | PI3 | 6.738762497 |
| NM_019618 | IL36G | 6.742761413 |
| NM_000576 | IL1B | 11.31259177 |
| NM_006664 | CCL27 | 2.97282529 |
| NM_000584 | IL8 | 2.258148839 |
| NM_000963 | PTGS2 | 2.284968542 |
| NM_001184779 | NOX5 | 3.303502622 |
| NM_000379 | XDH | 2.57463302 |

TABLE 4
Decreased genes

| Name | Gene symbol | Fold change |
|---|---|---|
| NM_004887 | CXCL14 | −12.19511071 |
| NM_003102 | SOD3 | −4.341912612 |
| NM_006121 | KRT1 | −3.259468923 |
| NR_002196 | H19 | −4.151100642 |
| NM_012114 | CASP14 | −2.396041041 |
| NM_000421 | KRT10 | −2.269122522 |
| NM_001080125 | CASP8 | −2.25127321 |
| NM_002275 | KRT15 | −4.343467673 |
| NM_002274 | KRT13 | −3.269661942 |

### EXAMPLE 5

### Real-Time RT-PCR

The normal human keratinocytes cultured in Example 2 were treated 12.5 μg of the microdust with a particle diameter of 2.5 μm extracted in Example 1 in 1 mL of a cell culture medium and relative mRNA expression level was measured using the primers described in Tables 5 and 6 (TaqMan® primers, Applied Biosystems).

TABLE 5
Increased genes

| Name | Gene symbol | TaqMan ® primer |
|---|---|---|
| NM_002963 | S100A7 | Hs00161488_m1 |
| NM_002964 | S100A8 | Hs00374263_m1 |
| NM_002965 | S100A9 | Hs00268204_m1 |
| NM_000499 | CYP1A1 | Hs00153120_m1 |
| NM_000104 | CYP1B1 | Hs00164383_m1 |
| NM_002638 | PI3 | Hs00160066_m1 |
| NM_019618 | IL36G | Hs00219742_m1 |
| NM_000576 | IL1B | Hs01555410_m1 |
| NM_006664 | CCL27 | Hs00171157_m1 |
| NM_000584 | IL8 | Hs00174103_m1 |
| NM_000963 | PTGS2 | Hs00153133_m1 |
| NM_001184779 | NOX5 | Hs00225846_m1 |
| NM_000379 | XDH | Hs00166010_m1 |

TABLE 6
Decreased genes

| Name | Gene symbol | TaqMan ® primer |
|---|---|---|
| NM_004887 | CXCL14 | Hs01557413_m1 |
| NM_003102 | SOD3 | Hs00162090_m1 |
| NM_006121 | KRT1 | Hs00196158_m1 |
| NR_002196 | H19 | Hs00262142_g1 |
| NM_012114 | CASP14 | Hs00201637_m1 |
| NM_000421 | KRT10 | Hs00166289_m1 |
| NM_001080125 | CASP8 | Hs01018151_m1 |
| NM_002275 | KRT15 | Hs00267035_m1 |
| NM_002274 | KRT13 | Hs02558881_s1 |

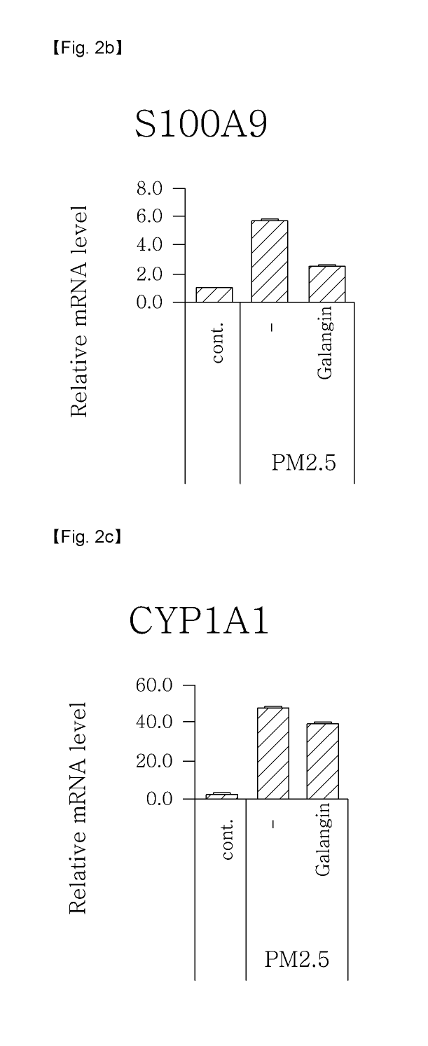

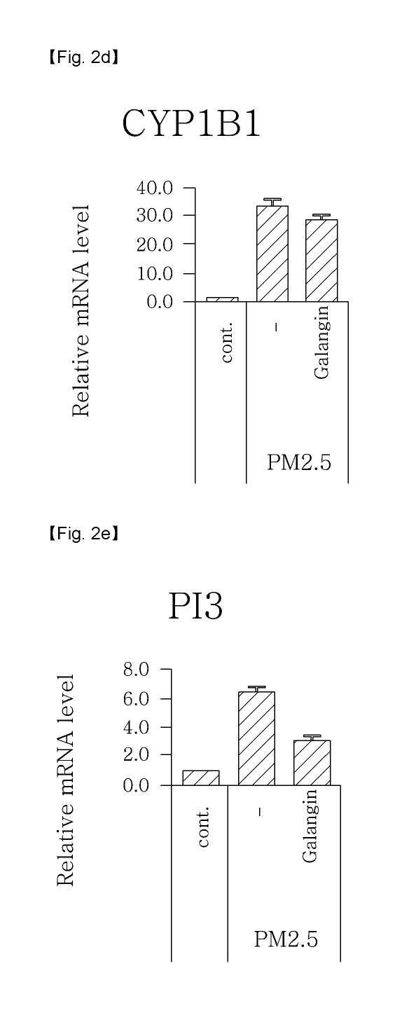

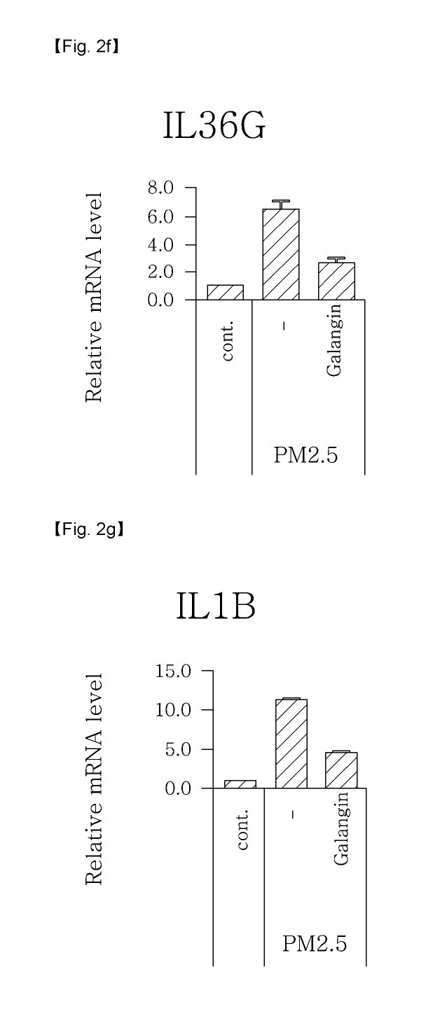

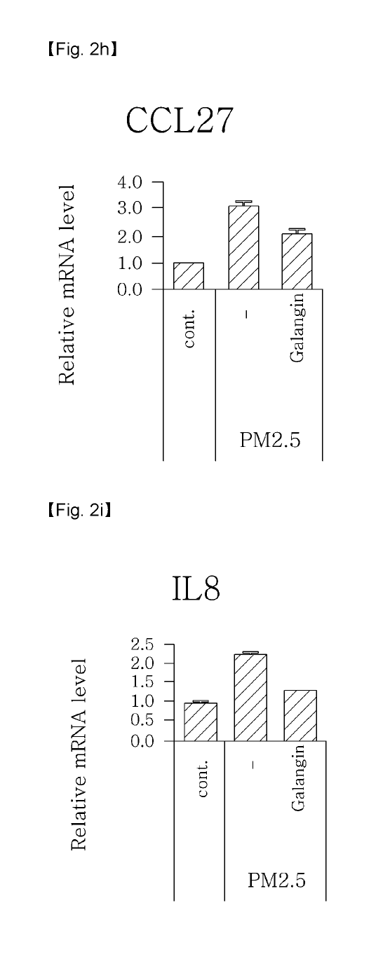

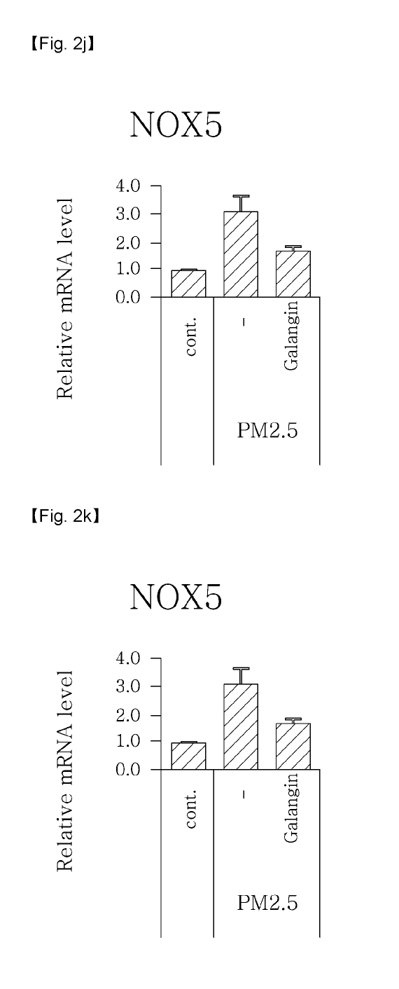

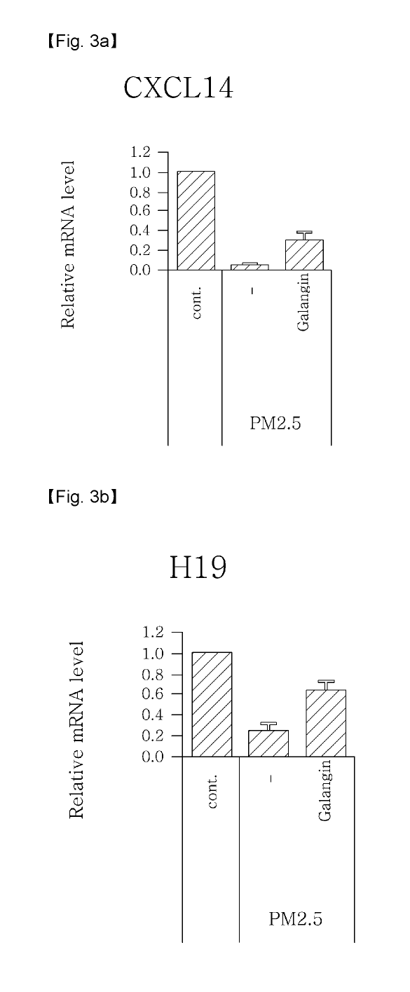

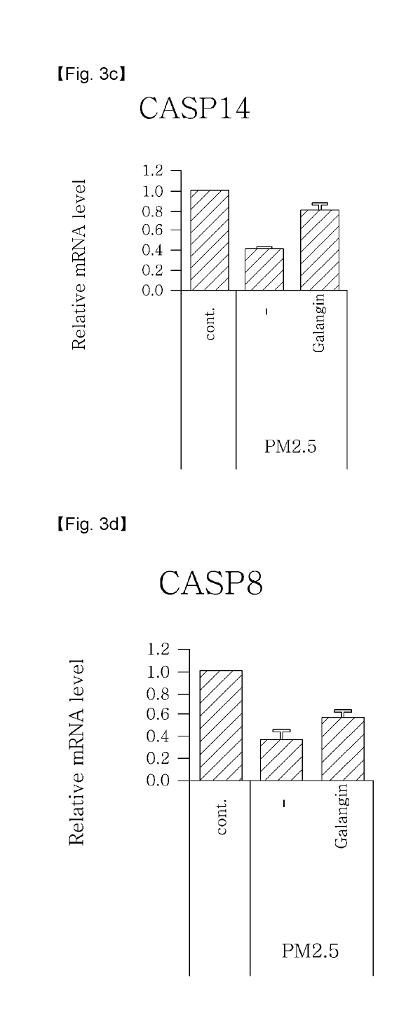

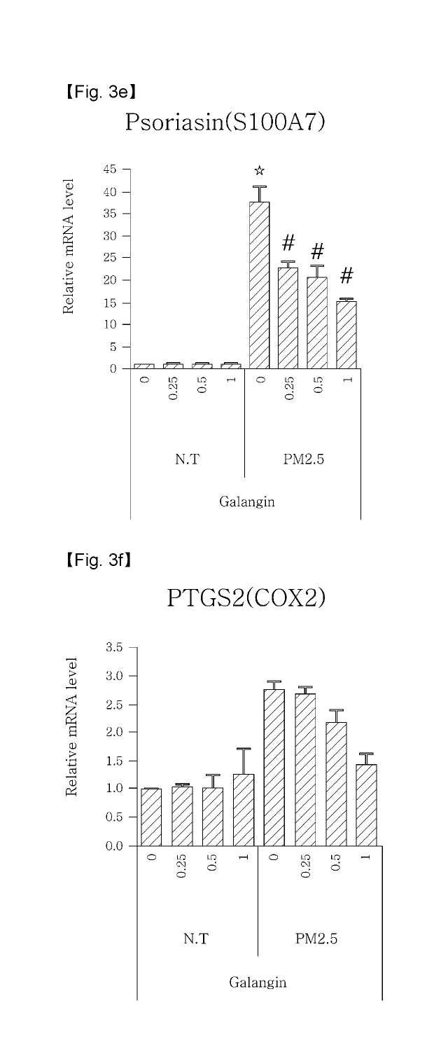

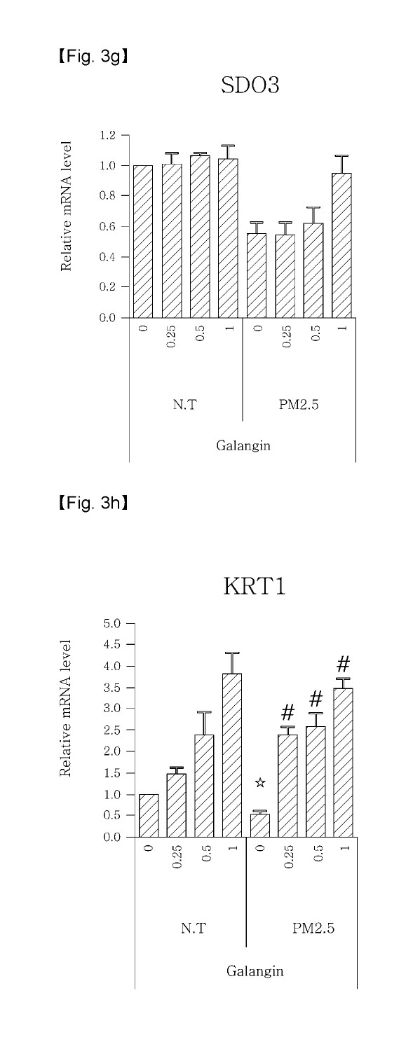

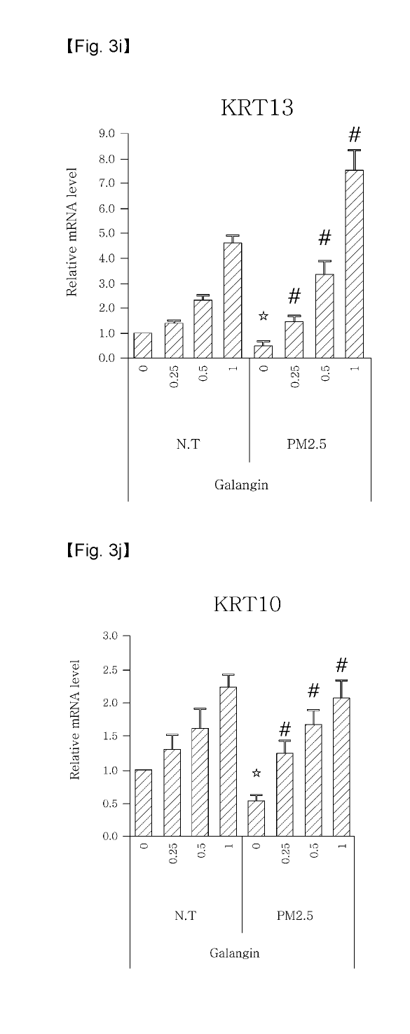

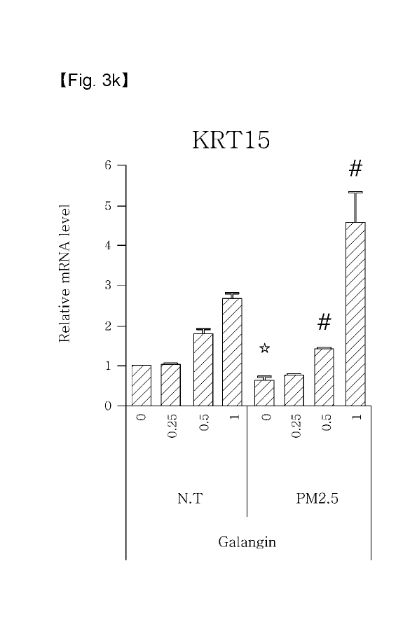

The microdust-treated normal human keratinocytes or the normal human keratinocytes cultured in Example 2 but not treated with microdust were treated with galangin at different concentrations (0.25 μM, 0.5 μM, 1 μM and 2 μM). 24 hours later, the culture medium was removed and the cells were washed with 2 mL of phosphate-buffered saline (PBS). Then, RNA was isolated from the cells using TRIzol reagent (Invitrogen, Carlsbad, Calif., USA). For S100A8, S100A9, CYP1A1, CYP1B1, PI3, IL36G, IL1B, CCL27, IL8, NOX6, XDH, CXCL14, H19, CASP14 and CASP8, expression level was measured after treating with 0.25 μM galangin. The galangin was purchased commercially from Nanjing Chemlin Chemical (CAS No. 548-83-4). The isolated RNA was purified once again with Qiagen RNA kit Qiagen, Valencia, Calif.) and the quality of RNA was determined using Agilent 2100 BioAnalyzer (Agilent Technologies, Santa Clara, Calif., USA). cDNA synthesized from the RNA using SuperScript reverse transcriptase (RT) kit (Invitrogen, Carlsbad, Calif.) was quantitatively analyzed by real time-reverse transcription polymerase chain reaction (Q-RT-PCR) using the primers described in Tables 5 and 6. The change in gene expression pattern was evaluated in real time using TaqMan gene expression assay kit (Applied Biosystems, Foster City, Calif.). The result is shown in FIGS. 2 and 3. The Q-RT-PCR and real-time PCR were conducted in accordance with the standard protocols of Life Technologies, specifically, 95° C. for 20 seconds followed by 40 cycles of 95° C. for 3 seconds and 60° C. for 30 seconds.

As seen from FIGS. 2 and 3, there were the genes whose expression was increased or decreased in the skin cells stimulated by microdust and returned to the normal level upon treatment with galangin.

### EXAMPLE 6

### Measurement of Change in Gene Expression in Normal Keratinocytes upon Treatment with Galangin

After treating the normal human keratinocytes cultured in Example 2 with galangin at different concentrations (0 μM, 0.5 μM, 1 μM and 2 μM), the relative mRNA expression level of filaggrin, keratin 10, keratin 1, keratin 13 and keratin 15 was measured.

24 hours after the treatment with galangin, the culture medium was removed and the cells were washed with 2 mL of phosphate-buffered saline (PBS). Then, RNA was isolated from the cells using TRIzol reagent (Invitrogen, Carlsbad, Calif., USA).

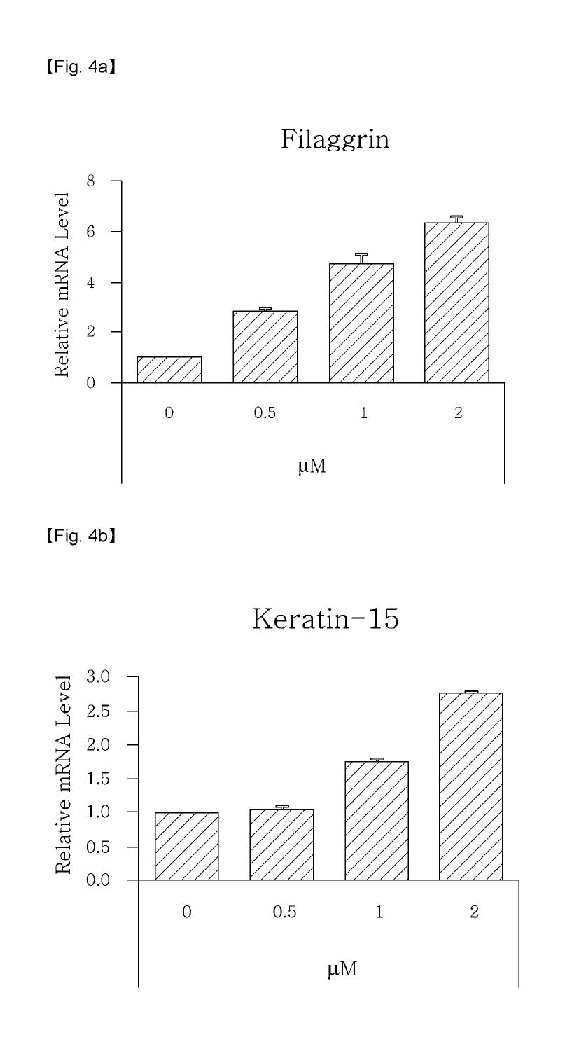

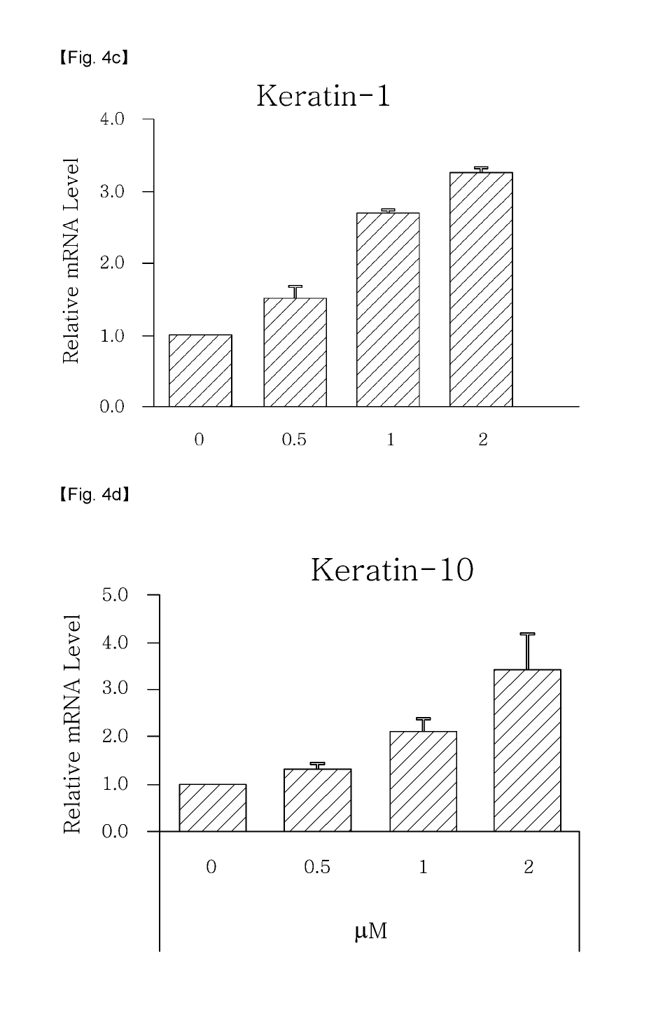

Subsequently, the change in gene expression was evaluated by real-time PCR in the same manner as in Example 5. The result is shown in FIG. 4. The primers used to amplify the genes are shown in Tables 5 and 6. For filaggrin, TaqMan® Hs00856927_g1 was used as the primer.

As seen from FIG. 4, filaggrin, keratin 10, keratin 1, keratin 13, keratin 15 showed increased expression with increasing galangin concentration even in the cells not treated with microdust.

### EXAMPLE 7

### Increased Differentiation of Keratinocytes upon Treatment with Galangin

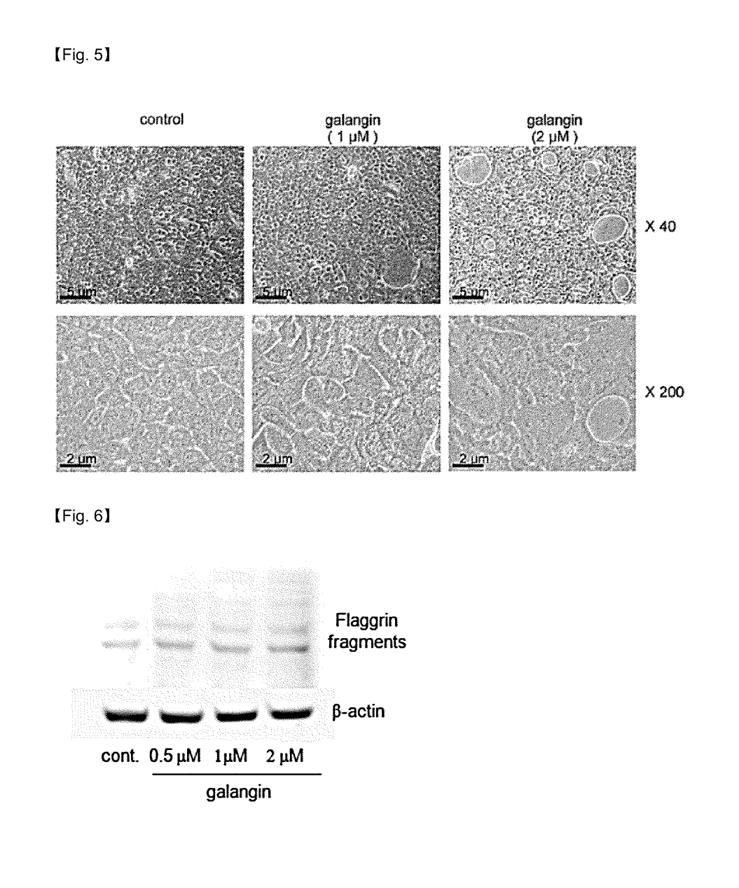

The normal human keratinocytes cultured in Example 2 were treated with galangin at different concentrations (0 μM, 1 μM and 2 μM). 24 hours later, the culture medium was removed and the degree of differentiation of the keratinocytes was observed under an optical microscope (Olympus IX71, x40 and x200). As seen from FIG. 5, the normal human keratinocytes not treated with microdust showed active differentiation with increasing galangin concentration.

### EXAMPLE 8

### Increased Expression of Filaggrin Protein upon Ttreatment with Galangin

The normal human keratinocytes cultured in Example 2 were treated with galangin at different concentrations (0 μM, 0.5 μM, 1 μM and 2 μM). 24 hours later, the culture medium was removed and the cells were washed with 2 mL of phosphate-buffered saline (PBS). After adding cell lysis buffer and vortexing, proteins were quantified from the obtained supernatant. The proteins obtained from the epidermis of normal skin and dry skin were loaded on SDS gel and then blotted using the filaggrin antibody (Covance, France). The quantification result was normalized to that of β-actin (Sigma, USA). As seen from FIG. 6, the expression of filaggrin protein increased with increasing galangin concentration.

Hereinafter, the present disclosure will be described in detail through formulation examples. However, the following formulation examples are for illustrative purposes only and the scope of the present disclosure is not limited by them.

### FORMULATION EXAMPLE 1

### Soap

TABLE 7

| Ingredients | Contents (%) |
|---|---|
| Galangin | 5.00 |
| Oil and fat | adequate |
| Sodium hydroxide | adequate |
| Sodium chloride | adequate |
| Fragrance | adequate |
| Purified water | balance |

### FORMULATION EXAMPLE 2

### Lotion

TABLE 8

| Ingredients | Contents (%) |
|---|---|
| Galangin | 5.00 |
| L-Ascorbic acid 2-phosphate magnesium salt | 1.00 |
| Water-soluble collagen (1% aqueous solution) | 1.00 |
| Sodium citrate | 0.10 |
| Citric acid | 0.05 |
| Licorice extract | 0.20 |
| 1,3-Butylene glycol | 3.00 |
| Purified water | balance |

### FORMULATION EXAMPLE 3

### Cream

TABLE 9

| Ingredients | Contents (%) |
|---|---|
| Galangin | 3.00 |
| Polyethylene glycol monostearate | 2.00 |
| Self-emulsifying glyceryl monostearate | 5.00 |
| Cetyl alcohol | 4.00 |
| Squalene | 6.00 |
| Glyceryl tri(2-ethylhexanoate) | 6.00 |
| Sphingolipid | 1.00 |
| 1,3-Butylene glycol | 7.00 |
| Purified water | balance |

### FORMULATION EXAMPLE 4

### Ointment

TABLE 10

| Ingredients | Contents (%) |
|---|---|
| Galangin | 5.00 |
| Polyvinyl alcohol | 13.00 |
| L-Ascorbic acid 2-phosphate magnesium salt | 1.00 |
| Lauroyl hydroxyproline | 1.00 |
| Water-soluble collagen (1% aqueous solution) | 2.00 |
| 1,3-Butylene glycol | 3.00 |
| Ethanol | 5.00 |
| Purified water | balance |

### FORMULATION EXAMPLE 5

### Cosmetic Solution

TABLE 11

| Ingredients | Contents (%) |
|---|---|
| Galangin | 3.00 |
| Hydroxyethyl cellulose (2% aqueous solution) | 12.00 |
| Xanthan gum (2% aqueous solution) | 2.00 |
| 1,3-Butylene glycol | 6.00 |
| Thick glycerin | 4.00 |
| Sodium hyaluronate (1% aqueous solution) | 2.00 |
| Purified water | balance |

### FORMULATION EXAMPLE 6

### Health Food

TABLE 12

| Ingredients | Contents | |
|---|---|---|
| Galangin | 2 | mg |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

### FORMULATION EXAMPLE 7

### Health Drink

TABLE 13

| Ingredients | Contents | |
|---|---|---|
| Galangin | 50 | mg |
| Citric acid | 1000 | mg |
| Oligosaccharide | 100 | g |
| Taurine | 1 | g |

| | |
|---|---|
| Purified water | balance |

1.-4. (canceled)

5. A method for diagnosing damage of skin cells or skin barrier by microdust comprising:
a) measuring the expression level of the mRNA of one or more gene selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27 (NM_006664) gene, IL8 (NM_000584) gene, PTGS2 (NM_000963) gene, NOX5 (NM_001184779) gene, XDH (NM_000379) gene, CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene and KRT13 (NM_002274) gene or protein encoded thereby, from a skin cell sample of a subject; and
b) comparing the expression level with the expression level of the mRNA of the gene or protein encoded thereby, in a skin cell sample not damaged by microdust.

6. The method for diagnosing damage of skin cells or skin barrier by microdust according to claim 5 further comprising determining that skin cells or skin barrier is damaged by microdust
1) when the expression level of the mRNA of one or more gene selected from a group consisting of CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene, KRT13 (NM_002274) gene and filaggrin gene or protein encoded thereby, measured from the skin cells of a subject is lower than that of a skin cell sample not damaged by microdust, or
2) when the expression level of the mRNA of one or more gene selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27 (NM_006664) gene, IL8 (NM_000584) gene, PTGS2 (NM_000963) gene, NOX5 (NM_001184779) gene and XDH (NM_000379) gene or protein encoded thereby measured from the skin cells of a subject is higher than that of a skin cell sample not damaged by microdust.

7. The method according to claim 5, wherein the expression level of the mRNA of the gene or protein is measured by one or more method selected from a group consisting of microarray, PCR, NGS (next-generation sequencing), western blot, northern blot, ELISA, radioimmunoassay, radioimmunodiffusion, histological immunostaining and immunoprecipitation assay.

8. A method for screening a substance improving skin damage by microdust comprising:
treating skin cells with microdust;
treating the microdust-treated skin cells with a test substance; and
checking the expression level of the mRNA of one or more gene selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27 (NM_006664) gene, IL8 (NM_000584) gene, PTGS2 (NM_000963) gene, NOX5 (NM_001184779) gene, XDH (NM_000379) gene, CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene and KRT13 (NM_002274) gene or protein encoded by the genes in the skin cells treated with the test substance, before and after the treatment with the test substance.

9. The method for screening a substance improving skin damage by microdust according to claim 8, further comprising determining the test substance as a substance improving skin damage by microdust
1) when the expression level of the mRNA of one or more gene selected from a group consisting of CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene, KRT13 (NM_002274) gene and filaggrin gene or protein encoded by the genes is increased after the treatment with the test substance as compared to before the treatment with the test substance, or
2) when the expression level of the mRNA of one or more gene selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27 (NM_006664) gene, IL8 (NM_000584) gene, PTGS2 (NM_000963) gene, NOXS (NM_001184779) gene and XDH (NM_000379) gene or protein encoded by the genes is decreased after the treatment with the test substance as compared to before the treatment with the test substance.

10. The method for screening a substance improving skin damage by microdust according to claim 8, wherein the skin cell is a keratinocyte.

11. A improving skin condition comprising administrating a composition comprising galangin, an isomer thereof, a pharmaceutically acceptable salt thereof, a prodrug thereof, a hydrate thereof or a solvate thereof as an effective ingredient to a subject need thereof.

12.-14. (canceled)

15. The method according to claim 11, wherein the composition comprises 0.000001-30 wt % of galangin, an isomer thereof, a pharmaceutically acceptable salt thereof, a prodrug thereof, a hydrate thereof or a solvate thereof based on the total weight of the composition.

16. The method according to claim 11, wherein the concentration of the galangin, the isomer thereof, the pharmaceutically acceptable salt thereof, the prodrug thereof, the hydrate thereof or the solvate thereof is 0.1-5 μM based on the total volume of the composition.

17. The method according to claim 11, wherein the composition promotes the expression of one or more selected from a group consisting of CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene, KRT13 (NM_002274) gene and filaggrin gene.

18. The method according to claim 11, wherein the composition decreases the expression of one or more selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27 (NM_006664) gene, IL8 (NM_000584) gene, PTGS2 (NM_000963) gene, NOXS (NM_001184779) gene and XDH (NM_000379) gene.

19. The method according to claim 11, wherein the composition promotes the synthesis of filaggrin protein or keratin protein.

20. The method according to claim 11, wherein the composition is a cosmetic composition, a pharmaceutical composition or a health food composition.

21. The method for diagnosing damage of skin cells or skin barrier by microdust according to claim 5, wherein the expression level of the mRNA of the gene or protein is measured by a composition for diagnosing damage of skin cells or skin barrier by microdust comprising an agent for measuring the expression level of mRNA of one or more gene selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27 (NM_006664) gene, IL8 (NM_000584) gene, PTGS2 (NM_000963) gene, NOX5 (NM_001184779) gene, XDH (NM_000379) gene, CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene and KRT13 (NM_002274) gene or protein thereof.

22. The method according to claim 21, wherein the agent for measuring the expression level of the mRNA or protein thereof is a polynucleotide complementary to the mRNA of the gene or a fragment thereof, or a probe or a primer capable of amplifying the gene.

23. The method according to claim 21, wherein the agent for measuring the expression level of the mRNA or protein thereof is an antibody specifically recognizing the protein.

24. The method for diagnosing damage of skin cells or skin barrier by microdust according to claim 5, wherein the expression level of the mRNA of the gene or protein is measured by a kit for diagnosing damage of skin cells or skin barrier by microdust comprising a composition comprising an agent for measuring the expression level of mRNA of one or more gene selected from a group consisting of S100A7 (NM_002963) gene, S100A8 (NM_002964) gene, S100A9 (NM_002965) gene, CYP1A1 (NM_000499) gene, CYP1B1 (NM_000104) gene, PI3 (NM_002638) gene, IL36G (NM_019618) gene, IL1B (NM_000576) gene, CCL27 (NM_006664) gene, 11,8 (NM_000584) gene, PTGS2 (NM_000963) gene, NOX5 (NM_001184779) gene, XDH (NM_000379) gene, CXCL14 (NM_004887) gene, SOD3 (NM_003102) gene, KRT1 (NM_006121) gene, H19 (NR_002196) gene, CASP14 (NM_012114) gene, KRT10 (NM_000421) gene, CASP8 (NM_001080125) gene, KRT15 (NM_002275) gene and KRT13 (NM_002274) gene or protein thereof.

25. The method according to claim 11, wherein the improving skin condition comprises moisturizing skin, enhancing skin barrier function, inducing differentiation of keratinocytes, or improving skin damage by microdust.
